(19) United States
(12) Patent Application Publication
(10) Pub. No.: US 2012/0136101 A1
(43) Pub. Date: May 31, 2012

(54) ESTER-BASED PLASTICIZER

(76) Inventors: Seung Gweon Hong, Yuseong-gu (KR); Kwon Tae Wook, Yuseong-gu (KR); Ki Nam Chung, Yuseong-gu (KR); Hao Wang, Jongro-gu (KR)

(21) Appl. No.: 13/384,799

(22) PCT Filed: Jul. 13, 2010
(86) PCT No.: PCT/KR10/04549

§ 371 Date: Jan. 19, 2012

(30) Foreign Application Priority Data
Jul. 20, 2009 (KR) 10-2009-00606056

(51) Int. Cl. C08K 5/12; C07C 67/26; C07C 69/78

(52) U.S. Cl. 524/308; 560/106; 560/112

(57) ABSTRACT

The present invention relates to a novel ester-based plasticizer, and, more particularly, to an ester plasticizer, which is synthesized by esterifying a polyhydric alcohol and an alkylene oxide with an aliphatic carboxylic acid or an aromatic carboxylic acid. When a polyvinylchloride resin is manufactured using the ester-based plasticizer, there are advantages in that products having a high plasticizing efficiency can be obtained and in that products having excellent physical properties, such as hardness, tensile strength, etc., can be obtained.

### TECHNICAL FIELD

The present invention relates to an ester plasticizer, which is synthesized by esterifying a polyhydric alcohol and an alkylene oxide with an aliphatic carboxylic acid or an aromatic carboxylic acid, and a method of manufacturing the same.

### BACKGROUND ART

A polyvinylchloride resin is a homopolymer of vinyl chloride or a heteropolymer including 50% or more of vinylchloride, and is a general-purpose resin which can be used in molding methods such as extrusion molding, injection molding, calendaring or the like. Polyvinylchloride resins are widely used to manufacture various products, such as pipes, electric wires, electric appliances, toys, films, sheets, artificial leathers, tarpaulins, tapes, food wrappers, medical appliances and the like, using the molding method. Such a polyvinylchloride resin can be imparted with various processing properties by suitably adding various additives such as a plasticizer, a stabilizer, a filler, a pigment and the like.

Among the additives, a plasticizer is an essential additive which serves to impart various physical properties and functions, such as workability, flexibility, an electrical insulation property, adhesivity and the like, to a polyvinylchloride resin by the addition thereof.

In the case of a plasticizer, low volatility is a very important factor, and is important both when it is mixed in a plastic composition and when it is practically used in molded products. Further, a plasticizer must be harmless to the health so that the plasticizer can be used in the fields of foods, drinks, medicals and the like. A phthalate-based plasticizer is a typical example of such a plasticizer.

However, owing to the dispute over the toxicity of regenerated plastics under laws regulating poisonous materials, it is predicted that the usage of the phthalate-based plasticizer will be remarkably reduced in the future. Therefore, it is required to develop a plasticizer including an ester compound containing no phthalate as a basic backbone and having a plasticizing efficiency equal to that of the phthalate-based plasticizer.

Japanese Unexamined Patent Publication No. 2005-154623 discloses a non-phthalate plasticizer. This non-phthalate plasticizer, which is an ester compound prepared from an alkylene oxide adduct of a polyhydric alcohol and a monocarboxylic acid, is a polymer prepared by making an alkylene oxide adduct and then reacting the alkylene oxide adduct with a carboxylic acid.

Japanese Unexamined Patent Publication No. 2001-114729 discloses a non-phthalate plasticizer. This non-phthalate plasticizer is a plasticizer prepared from ethylene glycol and a carboxylic acid. However, this non-phthalate plasticizer must include an entrainer as an essential element, and is prepared without using alkylene oxide.

There have been various attempts to prevent bleeding. For example, Japanese Unexamined Patent Publication Nos. 2005-232403 and 2005-112933 disclose a plasticizer including a polyhydric alcohol, an alkylene oxide and ester acetate. However, this plasticizer did not improve bleeding resistance without deteriorating transparency or flexibility.

### DISCLOSURE

### Technical Problem

The present invention intends to provide an ester-based plasticizer having physical properties equal to or higher than those of a conventional phthalate-based plasticizer, which is synthesized by mixing alkylene oxide with a polyhydric alcohole and then esterifying the mixture thereof with an aliphatic carboxylic acid or an aromatic carboxylic acid.

Further, the present invention intends to provide a method of manufacturing an ester-based plasticizer using a polyhydric acid, alkylne oxide and a carboxylic acid.

Further, the present invention intends to provide a polyvinylchloride resin including the ester-based plasticizer.

### Technical Solution

An aspect of the present invention provides an ester-based plasticizer, synthesized by esterifying a polyhydric alcohol and an alkylene oxide with an aliphatic carboxylic acid or an aromatic carboxylic acid.

Another aspect of the present invention provides a method of manufacturing an ester-based plasticizer by esterifying a polyhydric alcohol and an alkylene oxide with an aliphatic carboxylic acid or an aromatic carboxylic acid.

Still another aspect of the present invention provides a polyvinylchloride resin composition, comprising: 100 parts by weight of a polyvinylchloride resin; and 10˜150 parts by weight of the ester-based plasticizer.

### Advantageous Effects

When a polyvinylchloride resin composition is manufactured using the ester-based plasticizer of the present invention prepared by mixing alkylene oxide with a polyhydric alcohol and esterifying the mixture thereof with aliphatic acid or benzoic acid, there are advantages in that products having a high plasticizing efficiency can be obtained and in that products having excellent physical properties, such as weight loss on heating, etc., can be obtained.

### BEST MODE

The ester-based plasticizer according to an embodiment of the present invention is synthesized by esterifying a polyhydric alcohol and an alkylene oxide with an aliphatic carboxylic acid or an aromatic carboxylic acid.

The polyhydric alcohol is a substituted or unsubstituted alcohol of 3 to 15 carbon atoms including two or more hydroxy groups. For example, glycerin, cyclohexanediol, cyclohexanedimethanol, cyclopentanediol, 1,4-butanediol, 1,3-butanediol, pentaerythritol, sorbitol or the like may be used. Preferably, a dihydric alcohol of 3 to 6 carbon atoms may be used as the polyhydric alcohol.

The alkylene oxide may be ethylene oxide and/or propylene oxide but is not limited thereto. The alkylene oxide is used in an amount of 1˜5 mol based on 1 mol of a hydroxy group of a polyhydric alcohol. When the amount of the alkylene oxide is greater than 5 moles, the viscosity of the ester-based plasticizer becomes high, and thus workability becomes low. Further, when the amount thereof is less than 1 mol, desired effects are not exhibited. Preferably, the alkylene oxide is used in an amount of 1˜3 mol.

The carboxylic acid may be a substituted or unsubstituted aliphatic carboxylic acid of 1 to 12 carbon atoms or a substituted or unsubstituted aromatic or heterocyclic carboxylic acid of 5 to 11 carbon atoms. Examples of the carboxylic acid may include acetic acid, hexanoic acid, octanoic acid, 2-ethylhexanoic acid, nonanoic acid, decanoic acid, dodecanoic acid, benzoic acid and methyl benzodic acid, and, preferably, acetic acid, hexanoic acid, octanoic acid, and benzodic acid. The carboxylic acid is used in an amount of 1.0˜5.0 mol based on 1 mol of the polyhydric alcohol. When the amount of the carboxylic acid is greater than 5.0 moles, the economic efficiency of the ester-based plasticizer in a raw material becomes low. Further, when the amount thereof is less than 1.0 mol, the physical properties of the ester-based plasticizer deteriorate because a hydroxy group (—OH) remains. Preferably, the carboxylic acid may be used such that the molar ratio of the carboxylic acid to the polyhydric alcohol is 1: 1.0˜3.0.

The ester-based plasticizer of the present invention is synthesized by esterifying a polyhydric alcohol and an alkylene oxide with an aliphatic carboxylic acid or an aromatic carboxylic acid. The catalyst used in the esterification reaction may be a catalyst generally used in an esterification reaction. As the catalyst, an acid catalyst, such as sodium bisulfate, p-toluene sulfonic acid, sulfuric acid or the like, may be used, but is not limited thereto. The catalyst may be used in an amount of 0.2˜5 wt % based on the reaction mixture.

Meanwhile, Examples of the solvent used in the synthesis of the ester-based plasticizer of the present invention may include, but are not limited to, hexane, toluene, xylene, and 1,2,4-trimethyl benzene.

The esterification reaction can be conducted at a temperature of 100˜250° C., preferably 160˜250° C.

[symbol] er the esterification reaction, the unreacted acid and the acid catalyst may be neutralized by the addition of an alkaline reagent. As the alkaline reagent, a sodium carbonate solution or a calcium carbonate solution may be used.

The reaction product is phase-separated to obtain a crude ester compound, and the crude ester compound is washed with water, dewatered and then filtered to obtain a product.

An embodiment of the present invention provides an ester-based plasticizer represented by the following Chemical Formula 1:

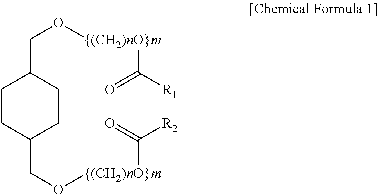

wherein R₁ and R₂ are each independently a substituted or unsubstituted straight-chain alkyl group of 1 to 20 carbon atoms, a substituted or unsubstituted alkenyl group of 2 to 20 carbon atoms, a substituted or unsubstituted cycloalkyl group of 3 to 20 carbon atoms, or a substituted or unsubstituted aryl or heteroaryl group of 5 to 10 carbon atoms; and n is an integer of 2˜4, and m is an integer of 1˜5.

Here, the molar ratio of carboxylic acid to 1,4-cyclohexane dimethanol is 1: 1.0˜5.0, preferably 1:1.0˜3.0.

Another embodiment of the present invention provides an ester-based plasticizer represented by the following Chemical Formula 2:

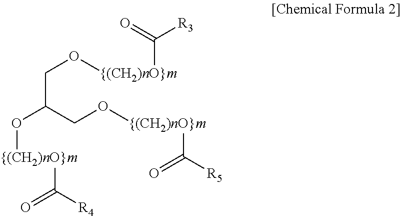

wherein R₃ to R₅ are each independently a substituted or unsubstituted straight-chain alkyl group of 1 to 20 carbon atoms, a substituted or unsubstituted alkenyl group of 2 to 20 carbon atoms, a substituted or unsubstituted cycloalkyl group of 3 to 20 carbon atoms, or a substituted or unsubstituted aryl or heteroaryl group of 5 to 10 carbon atoms; and n is an integer of 2˜4, and m is an integer of 1˜5.

Still another embodiment of the present invention provides an ester-based plasticizer represented by the following Chemical Formula 3:

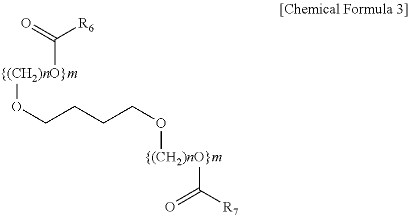

wherein R₆ and R₇ are each independently a substituted or unsubstituted straight-chain alkyl group of 1 to 20 carbon atoms, a substituted or unsubstituted alkenyl group of 2 to 20 carbon atoms, a substituted or unsubstituted cycloalkyl group of 3 to 20 carbon atoms, or a substituted or unsubstituted aryl or heteroaryl group of 5 to 10 carbon atoms; and n is an integer of 2˜4, and m is an integer of 1˜5.

Still another embodiment of the present invention provides a polyvinylchloride resin composition, including: 100 parts by weight of a polyvinylchloride resin; and 10˜150 parts by weight of the ester-based plasticizer. When the amount of the ester-based plasticizer is less than 10 parts by weight, there is a problem in that the polyvinylchloride resin composition does not have physical properties, such as plasticizing efficiency, adhesivity, etc. Further, when the amount thereof is greater than 150 parts by weight, there is a problem in that the thermal stability and migration resistance of the polyvinylchloride resin composition become poor.

The ester-based plasticizer of the present invention may be applied to chlorine-containing resins, such as chlorinated polyvinyl chloride, polyvinylidene chloride, chlorinated polyethylene, a vinyl chloride-vinyl acetate copolymer, a vinyl chloride-ethylene copolymer, a vinyl chloride-propylene copolymer, a vinyl chloride-styrene copolymer, a vinyl chloride-isobutylene copolymer, a vinyl chloride-vinylidene chloride copolymer, vinyl chloride-vinyl ether copolymers, and blends thereof; and synthetic resins containing no chlorine, such as an acrylonitrile-styrene copolymer, an acrylonitrile-styrene-butadiene terpolymer, an ethylene-vinyl acetate copolymer, polyester, blends thereof, block copolymers thereof, and graft copolymers thereof.

The polyvinylchloride resin composition may include 10˜150 parts by weight of the ester-based plasticizer based on 100 parts by weight of the polyvinylchloride resin.

The amount of the ester-based plasticizer of the present invention in the polyvinylchloride resin composition may be appropriately increased and decreased depending on the use of the polyvinylchloride resin composition. When the amount of the ester-based plasticizer is less than 10 parts by weight, the polyvinylchloride resin composition cannot obtain flexibility or workability, which can be exhibited by the ester-based plasticizer. Further, when the amount thereof is greater than 150 parts by weight, the polyvinylchloride resin composition cannot easily obtain desired mechanical properties, and can be eluted, which are not preferable. Meanwhile, the method of manufacturing the polyvinylchloride resin composition is not particularly limited, and the polyvinylchloride resin composition may be manufactured by any method well known to those skilled in the art.

The polyvinylchloride resin composition including the ester-based plasticizer according to the present invention may be used to manufacture: building materials, such as wall-finishing materials, flooring materials, window frames, wall papers, etc.; wire coating materials; interior and exterior materials for automobiles; agricultural materials such as vinyl houses, tunnels, etc.; food wrappers; film forming agents, such as sealant, plastisol, paint, ink, etc.; miscellaneous goods, such as synthetic leathers, coated fabrics, hoses, pipes, sheets, toys for infants, gloves, etc.; and the like.

### MODE FOR INVENTION

Hereinafter, the present invention will be described in more detail with reference to the following Examples and Comparative Examples. However, these Examples are set forth to illustrate the present invention, and the scope of the present invention is not limited thereto. In these Examples and Comparative Examples, physical properties were evaluated as follows.

### Hardness

Based on ASTM D2240, one point of a sample was pressed by a needle of a hardness tester (A Type) for 5 seconds, and the hardness value of the sample was measured. The hardness values of three points of each sample were measured, and then the average value of the hardness values was obtained. Hardness is used as an index for the plasticizing efficiency.

Tensile strength, elongation, modulus at 100% elongation

The tensile strength, elongation and modulus at 100% elongation of a sample were measured using a UTM, based on ASTM D412. A dumbbell-shaped sample was pulled at a crosshead speed of 200 mm/min, and then the tensile strength, elongation and modulus at 100% elongation of the cut point of the dumbbell-shaped sample were measured. The modulus at 100% elongation thereof is the tensile strength at 100% elongation thereof, and is related closely to the plasticizing efficiency.

### Weight Loss on Heating

A predetermined amount of a sample was left in a 180° C. oven for 24 hours, and the change in the weight thereof was measured.

### Example 1

### Preparation of an Ester-Based Plasticizer Using 1,4-Cyclohexanedimethanol Provided with 1 Mol of Ethylene Oxide (EO), Benzoic Acid and N-Hexanoic Acid

First, 1.0 mol of 1,4-cyclohexanedimethanol provided with 1 mol of EO, 0.6 mol of benzoic acid, 0.6 mol of n-hexanoic acid, 200 g of toluene (solvent), and 3.0 g of sodium bisulfate (catalyst) were put into a 2 L round flask provided with a stirrer and a condenser and then heated to 100° C., and then the reaction was conducted for 12 hours.

After the reaction, unreacted acid was removed by reducing the pressure to 5 mmHg at 200° C. using a vacuum pump, and a reaction product was neutralized using a sodium carbonate aqueous solution (10 wt %), washed with water, dewatered and then filtered using an adsorbent to obtain an ester-based plasticizer composition. The obtained ester-based plasticizer composition is a mixture including the compound represented by Formula 1 above as a main component.

### Preparation of a Polyvinylchloride Resin Composition

In order to evaluate the performance of the obtained ester plasticizer, a test sample was fabricated. That is, 100 parts by weight of a polyvinylchloride resin (LS-100, manufactured by LG Chem, Ltd.), 50 parts by weight of the ester-based plasticizer composition including the compound represented by Formula 1 above as a main component, and 1 part by weight of a stabilizer (LFX-1100, manufactured by Korea Daehyup Chem, Ltd.) were mixed, preheated to 185° C. for 1 minute, pressed for 1.5 minutes and then cooled for 2 minutes to form a sheet having a thickness of 2 mm. Then, dumbbell-shaped test samples were fabricated using the sheet.

The above-mentioned test was conducted using the plasticizer and test samples, and the results thereof are shown in Table 1 below.

### Example 2

### Preparation of an Ester-Based Plasticizer Using 1,4-Cyclohexanedimethanol Provided with 2 Mol of EO, Benzoic Acid and Acetic Acid

A plasticizer and a polyvinylchloride composition were prepared in the same manner as Example 1, except that 1,4-cyclohexanedimethanol provided with 2 mol of EO was used instead of 1,4-cyclohexanedimethanol provided with 1 mol of EO, and acetic acid was used instead of n-hexanoic acid. The test results thereof are shown in Table 1 below.

### Example 3

### Preparation of an Ester-Based Plasticizer Using Glycerol Provided with 1 Mol of EO, Benzoic Acid, N-Hexanoic Acid and Acetic Acid

A plasticizer and a polyvinylchloride composition were prepared in the same manner as Example 1, except that glycerol provided with 1 mol of EO was used instead of 1,4-cyclohexanalimethanol provided with 1 mol of EO, and acetic acid was used in addition to n-hexanoic acid. The test results thereof are shown in Table 1 below.

### Example 4

### Preparation of an Ester-Based Plasticizer Using Glycerol Provided with 2 Mol of EO, Benzoic Acid and Acetic Acid

A plasticizer and a polyvinylchloride composition were prepared in the same manner as Example 1, except that glycerol provided with 2 mol of EO was used instead of glycerol provided with 1 mol of EO, and acetic acid was doubly used instead of n-hexanoic acid. The test results thereof are shown in Table 1 below.

### Example 5

### Preparation of an Ester-Based Plasticizer Using 1,4-Butanediol Provided with 1 Mol of EO, Benzoic Acid and N-Octanoic Acid

A plasticizer and a polyvinylchloride composition were prepared in the same manner as Example 1, except that 1,4-butanediol provided with 1 mol of EO was used instead of 1,4-cyclohexanedimethanol provided with 1 mol of EO, and n-octanoic acid was used instead of n-hexanoic acid. The test results thereof are shown in Table 1 below.

### Example 6

### Preparation of an Ester-Based Plasticizer Using 1,4-Butanediol Provided with 2 Mol of EO, Benzoic Acid and Acetic Acid

A plasticizer and a polyvinylchloride composition were prepared in the same manner as Example 1, except that 1,4-butanediol provided with 2 mol of EO was used instead of 1,4-cyclohexanedimethanol provided with 1 mol of EO, and acetic acid was used instead of n-hexanoic acid. The test results thereof are shown in Table 1 below.

### Comparative Example 1

Test examples were fabricated using di-2-ethylhexyl phthalate, which is most widely used, as a plasticizer in the same manner as Example 1. The above-mentioned test was conducted using the test samples, and the results thereof are shown in Table 1 below.

### Comparative Example 2

Test examples were fabricated using diisononyl phthalate, which is increasingly used as an alternative to di-2-ethylhexyl phthalate, as a plasticizer in the same manner as Example 1. The above-mentioned test was conducted using the test samples, and the results thereof are shown in Table 1 below.

### Comparative Example 3

A plasticizer and a polyvinylchloride composition were prepared in the same manner as Example 1, except that 1,4-butanediol was used instead of 1,4-butanediol provided with 1 mol of EO. The test results thereof are shown in Table 1 below.

TABLE 1

| | | | | | | | Comp. | Comp. | Comp. |
|---|---|---|---|---|---|---|---|---|---|
| Measured items | Ex. 1 | Ex. 2 | Ex. 3 | Ex. 4 | Ex. 5 | Ex. 6 | Ex. 1 | Ex. 2 | Ex. 3 |
| Hardness, Shore A | 80 | 80 | 82 | 84 | 80 | 79 | 81 | 84 | 78 |
| Tensile strength, Kgf/cm² | 185 | 180 | 215 | 220 | 180 | 195 | 189 | 193 | 175 |
| Elongation, % | 390 | 394 | 402 | 398 | 380 | 360 | 370 | 373 | 400 |
| Modulus, Kgf/cm² | 83 | 81 | 83 | 90 | 83 | 101 | 85 | 96 | 80 |
| Weight loss on heating, % | 2 | 2 | 2 | 1 | 3 | 3 | 4 | 3 | 9 |

From the results shown in Table 1 above, it can be seen that the plasticizing efficiencies of the plasticizers of Examples 1 to 6 are equal to or higher than those of the most general plasticizers of Comparative Examples 1 and 2, and that the physical properties, such as weight loss on heating, etc., of the plasticizers of Examples 1 to 6 are equal to or higher than those of the most general plasticizers of Comparative Examples 1 and 2. Further, from the results of Example 5 and Comparative Example 3, it can be seen that, although the molecular weight of the plasticizer of Example 5, which is provided with EO, is higher than that of the plasticizer of Comparative Example 3, which is not provided with EO, the hardness of the plasticizer of Example 5 is not greatly high compared to that of the plasticizer of Comparative Example 3 and is similar to that of the plasticizer of Comparative Example 1, but the weight loss on heating of the plasticizer of Example 5 is improved compared to that of the plasticizer of Comparative Example 3.

Hardness is related closely to plasticizing efficiency. Therefore, the fact that the Hardness of the plasticizer does not become high greatly means that the compatibility of the plasticizer with a resin is maintained. Further, it is predicted that the plasticizer of the present invention generate a small amount of fume even when high-temperature treatment is conducted in the process of preparing the plasticizer because the weight loss on heating of the plasticizer becomes low.

Therefore, since the novel plasticizer of the present invention has high plasticizing efficiency, it is suitable for various types of molding processes depending on various uses, and it can be utilized in various fields.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

1. An ester-based plasticizer, synthesized by esterifying a polyhydric alcohol and an alkylene oxide with an aliphatic carboxylic acid or an aromatic carboxylic acid.

2. The ester-based plasticizer according to claim 1, wherein the polyhydric alcohol is a substituted or unsubstituted alcohol of 3 to 15 carbon atoms including two or more hydroxy groups.

3. The ester-based plasticizer according to claim 1, wherein the polyhydric alcohol is glycerin, 1,4-cyclohexanedimethanol or 1,4-butanediol.

4. The ester-based plasticizer according to claim 1, wherein the ester-based plasticizer is represented by Chemical Formula 1 below: 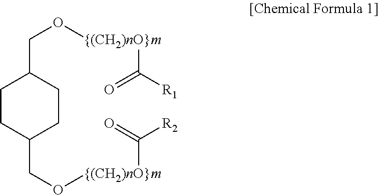
wherein R1 and R2 are each independently a substituted or unsubstituted straight-chain alkyl group of 1 to 20 carbon atoms, a substituted or unsubstituted alkenyl group of 2 to 20 carbon atoms, a substituted or unsubstituted cycloalkyl group of 3 to 20 carbon atoms, or a substituted or unsubstituted aryl or heteroaryl group of 5 to 10 carbon atoms; and n is an integer of 2˜4, and m is an integer of 1˜5.

5. The ester-based plasticizer according to claim 1, wherein the ester-based plasticizer is represented by Chemical Formula 2 below: 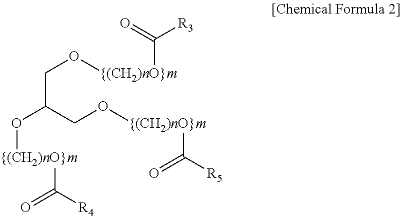
wherein R3 to R5 are each independently a substituted or unsubstituted straight-chain alkyl group of 1 to 20 carbon atoms, a substituted or unsubstituted alkenyl group of 2 to 20 carbon atoms, a substituted or unsubstituted cycloalkyl group of 3 to 20 carbon atoms, or a substituted or unsubstituted aryl or heteroaryl group of 5 to 10 carbon atoms; and n is an integer of 2˜4, and m is an integer of 1˜5.

6. The ester-based plasticizer according to claim 1, wherein the ester-based plasticizer is represented by Chemical Formula 3 below: 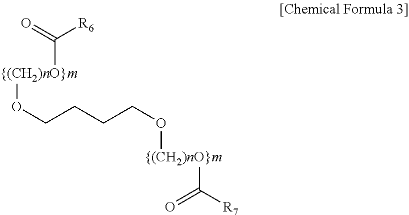
wherein R6 and R7 are each independently a substituted or unsubstituted straight-chain alkyl group of 1 to 20 carbon atoms, a substituted or unsubstituted alkenyl group of 2 to 20 carbon atoms, a substituted or unsubstituted cycloalkyl group of 3 to 20 carbon atoms, or a substituted or unsubstituted aryl or heteroaryl group of 5 to 10 carbon atoms; and n is an integer of 2˜4, and m is an integer of 1˜5.

7. A method of manufacturing an ester-based plasticizer by esterifying a polyhydric alcohol and an alkylene oxide with an aliphatic carboxylic acid or an aromatic carboxylic acid.

8. The method of manufacturing an ester-based plasticizer according to claim 7, wherein the alkylene oxide is used in an amount of 1˜5 mol based on 1 mol of a hydroxy group of a polyhydric alcohol.

9. The method of manufacturing an ester-based plasticizer according to claim 7, wherein the carboxylic acid is used in an amount of 1.0˜5.0 mol based on 1 mol of a polyhydric alcohol.

10. A polyvinylchloride resin composition, comprising:
100 parts by weight of a polyvinylchloride resin; and
10˜150 parts by weight of the ester-based plasticizer of claim 1.
